(19) United States
(12) Patent Application Publication
(10) Pub. No.: US 2019/0357992 A1
(43) Pub. Date: Nov. 28, 2019

(54) IMMOBILIZATION SYSTEM HAVING BITE-BLOCK STABILIZATION AND METHOD OF USING SAME

(71) Applicant: Klarity Medical Products, LLC, Newark, OH (US)

(72) Inventors: Matthew P. Calhoun, Newark, OH (US); Mary Ellen Devault, Newark, OR (US)

(21) Appl. No.: 15/989,379

(22) Filed: May 25, 2018

(51) Int. Cl. A61B 90/16; A61N 5/10; A61C 9/00; A61B 90/18; A61B 13/00

(52) U.S. Cl. CPC A61B 90/16; A61N 5/10; A61N 2005/1097; A61B 90/18; A61B 13/00; A61C 9/00

(57) ABSTRACT

An immobilization system, especially for use on the human head and neck area, is described in various embodiments. The system may include a bite block immobilization system having a bite block with a dental interface releasably engageable with a plurality of teeth of a patient, and a retention mask. The dental interface releasably engages the teeth of a patient and allows very highly accurate repositioning during multiple procedures. In some embodiments, the bite block may have an integral bite block airway passage, allowing a patient to have unobstructed breathing both during molding of the retention mask and during subsequent use of the system, and may include a tongue diverter, that may, by way of example only and not to limitation, be used to divert the tongue away from a therapeutic radiation beam. Steps for utilizing the system are further described.

### CROSS-REFERENCE TO RELATED APPLICATIONS

Not applicable.

### STATEMENT REGARDING FEDERALLY SPONSORED RESEARCH OR DEVELOPMENT

Not applicable.

### TECHNICAL FIELD

The present disclosure relates generally to an immobilization system, in particular, for making an anatomically high-accuracy moldable immobilization system having a bite-block stabilization feature, well-suited for patient immobilization during radiotherapy or other applications requiring a high degree of accuracy in patient positioning. This is of particular importance for radiotherapy of the bead and neck region.

### BACKGROUND OF THE INVENTION

The present invention relates to an immobilization system, for persons whose bodies or body parts are required to be retained in a particular position or attitude. In particular, a need exists for a simple and easy to use immobilization system that allows repetitive patient positioning with a high degree of accurate repeatability across time, for example, wherein radiation treatments must be directed at a certain anatomical point across different times. Such precise positioning of the head and neck area, as might be required for radiotherapy of that region, is particularly problematic due to difficulty in immobilizing the area, while maintaining the patient's ability to breath, and with a reasonable degree of patient comfort.

The use of low-temperature thermoplastics for patient positioning is well-known and dates back to splinting devices invented in the 1960's (Larson, U.S. Pat. No. 5,540,876). Splints are heated, usually in hot water, to a temperature of about 160° F., whereupon they become pliable and can be molded by hand directly on the patient's body part. These devices are well known in the field of occupational therapy and include splints with padding or cushioning material laminated to the thermoplastic to provide comfort against the patient's skin.

Plastic materials have been successfully used in the past for making splints, casts and the like. U.S. Pat. No. 3,490,444 describes the use of thermoplastic polydienes like transpolyisoprene and transpolychloroprene, which melt between 140° F. (60° Celsius) and 212° F. (100° Celsius), and which harden by crystallization at about 140° F. (60° Celsius), such that these plastics can be formed for use as a body supporting member. Poly (epsilon-caprolactone) (PCL) has also been found to be an excellent splint or cast material (U.S. Pat. No. 4,144,223). Polyurethanes based on prepolymers of poly (epsilon-caprolactone) have also been used (U.S. Pat. No. 4,316,457).

As described in earlier patents, the polymers can be heated in hot water at a temperature usually exceeding 122° F. (50° Celsius) and up to about 212° F. (100° Celsius), whereby they become soft, self-adherent and sufficiently pliable to be deformed and shaped as a cast, splint or protective device. When allowed to cool in air to about 140° F. (40° Celsius), the materials will remain pliable, moldable and cohesive for a period of several minutes, exhibiting a hysteresis, as described in U.S. Pat. No. 3,490,444. During this time the splint, cast or device can be molded directly to the patient without discomfort, and the shaped plastic sets hard by crystallization to assume a rigid form as a useful body support member or protective device.

In the field of radiation therapy, precise patient positioning is essential for treatment accuracy. An additional requirement is that patients be precisely re-positioned for repeated radiation treatments. Repositioning to an accuracy within millimeters, or even less, is generally desirable. This requires the positioning to be reproduced accurately each time the patient undergoes a treatment. Low-temperature thermoplastic masks in conjunction with other positioning methods are often used for such positioning. Masks are heated to a temperature of about 160° F. (71° C.), and formed directly on to the patient's head or other body part. The masks may be affixed to a table supporting the patient and cooled to form a firm mask holding the patient steady for treatment. After treatment, the mask may be removed. When the patient returns for the next treatment, the mask is releasably reattached, holding the patient in a reproduced position for treatment.

Various masks are used for radiation therapy treatments, including stereotactic head masks holding the top and bottom of the patient's head (Vilsmeier, U.S. Pat. No. 5,702,406). In the field of moldable head and neck masks, a particular problem is holding the mask, when heated, to conformance around the patient's facial and head area for the short period of time required for the mask to cool and assume a stable shape. A tension exists between holding the mask accurately to conform to these features, while maintaining a reasonable degree of patient comfort.

Another goal of patient immobilization systems for radiation therapies is the avoidance of as much mass surrounding the patient's body as possible, as the mass tends to attenuate a radiotherapy beam. Therefore, the avoidance of unnecessary bulk in the immobilization system confers an advantage over more bulky systems. However, at the same time, systems have found it difficult to achieve adequate stabilization without using relatively high-mass immobilization devices.

### SUMMARY OF THE INVENTION

The disclosed invention relates to methods to for making an immobilization system. Especially for those systems intended for use on the human head and neck area, there may be a retention mask, molded to the face or head contours of the patient, holding the desired body parts firmly to the formable support. This allows for high reproducibility in positioning.

The system may include a bite block immobilization system having a bite block with a dental interface releasably engageable with a plurality of teeth of a patient. The dental interface may be supplied with the system, or may be added at the time of use by a user. The dental interface releasably engages the teeth of a patient and allows very highly accurate repositioning during multiple procedures. In some embodiments, the bite block may have an integral bite block airway passage, allowing a patient to have unobstructed breathing both during molding of the retention mask and during subsequent use of the system.

In yet another series of embodiments, the bite block releasably cooperates at a tongue diverter receiver with a tongue diverter, that may, by way of example only and not limitation, be used to divert the tongue away from a therapeutic radiation beam. Steps for employing the system are further described below.

### BRIEF DESCRIPTION OF THE ILLUSTRATIONS

Without limiting the scope of the as disclosed herein and referring now to the drawings and figures:

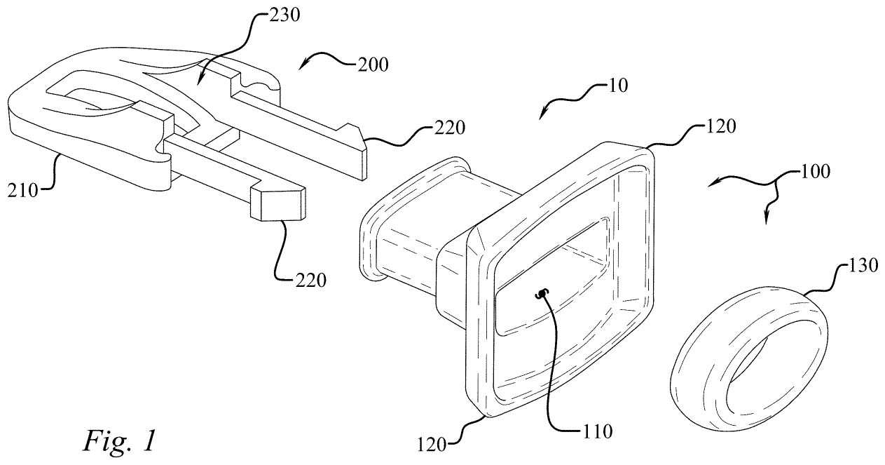

FIG. 1 is an unassembled elevated perspective view of an embodiment of the current specification;

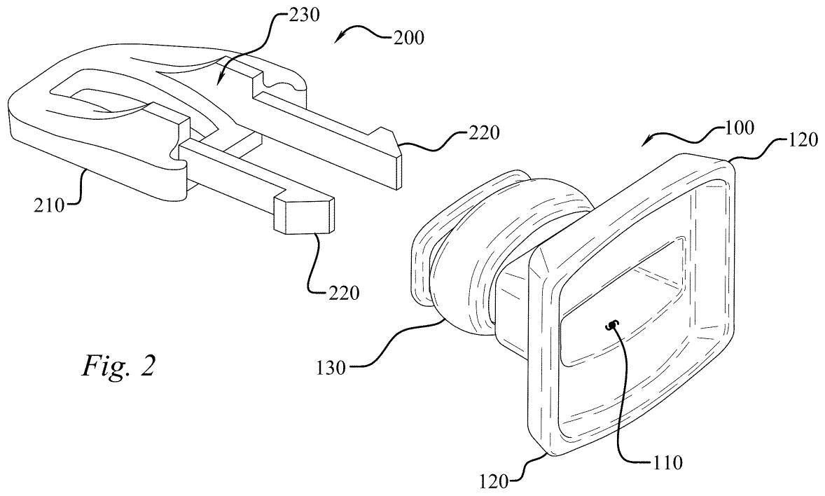

FIG. 2 is an unassembled elevated perspective view of a detail of a portion of the embodiment of FIG. 1;

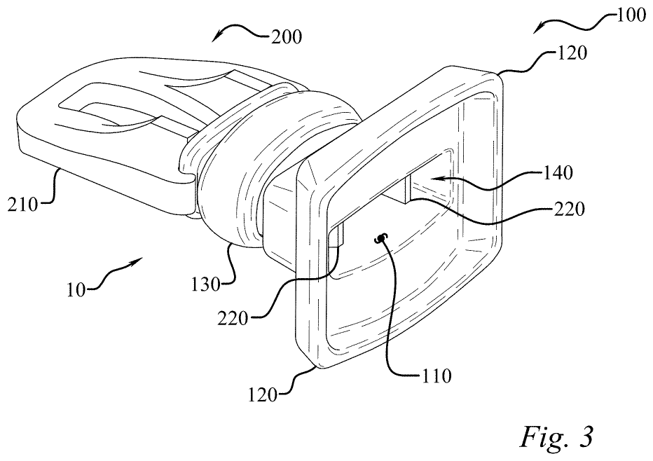

FIG. 3 is an assembled elevated perspective view of a further detail of a portion of the embodiment of FIG. 1;

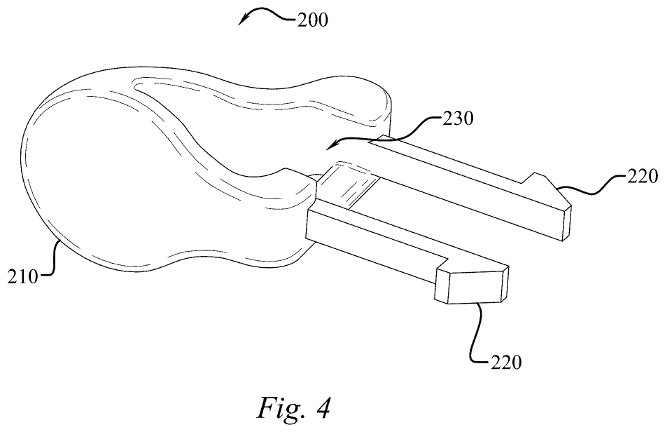

FIG. 4 is a further elevated perspective view of a detail of a portion of the embodiment of FIG. 1;

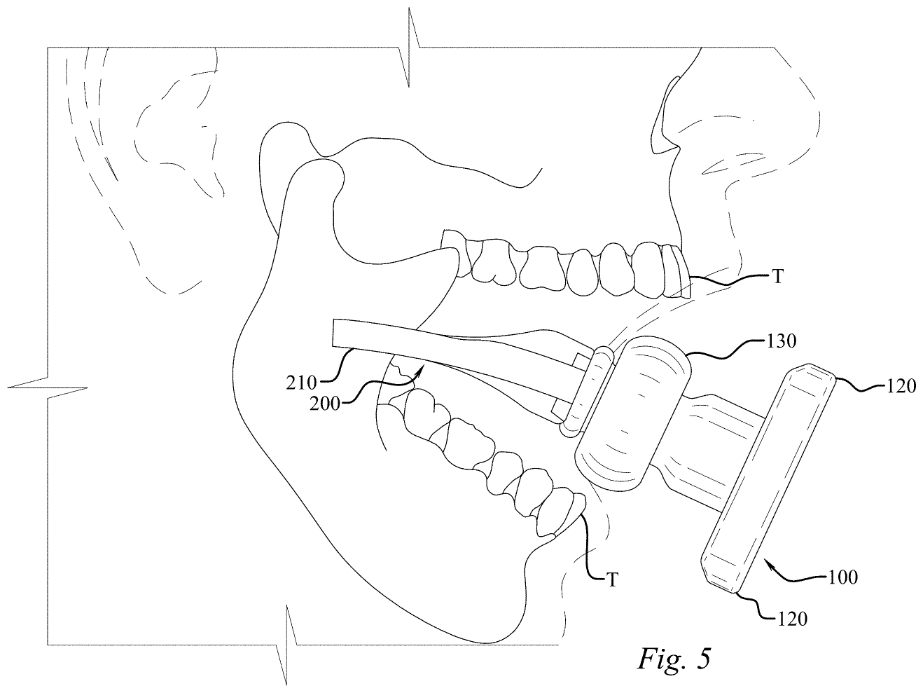

FIG. 5 is a lateral elevation view of an assembled embodiment of the device of FIG. 1 seen being placed in operating position between a patient's mandible and maxilla;

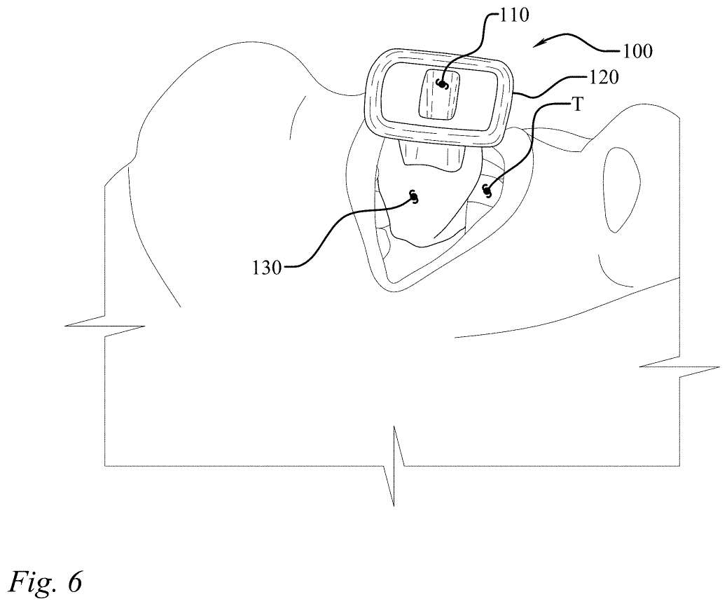

FIG. 6 is an elevated perspective view of an embodiment of the device of FIG. 1 in place in an operating position between a patient's mandible and maxilla;

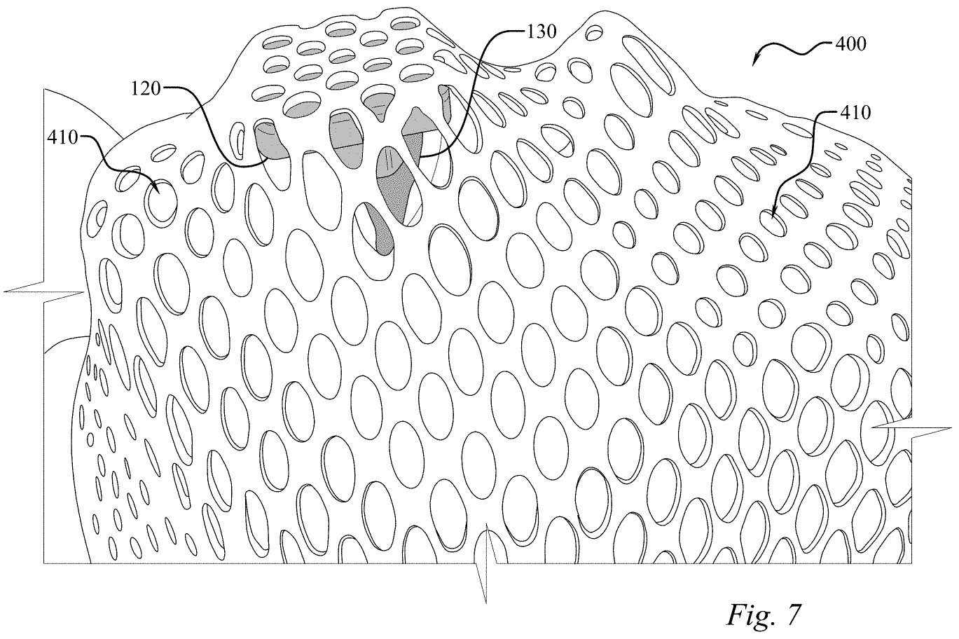

FIG. 7 is an elevated perspective view of a prior-art facial positioning mask placed over the embodiment of the device of FIG. 1 in an operating position;

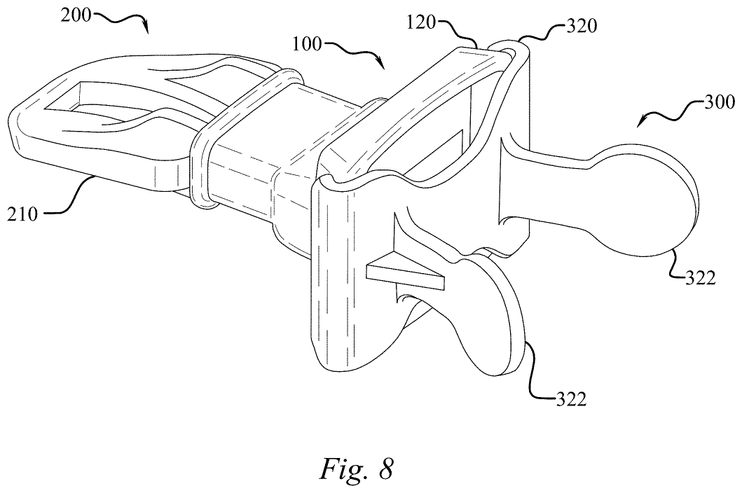

FIG. 8 is a further embodiment of the device of FIG. 1, further showing a digital manipulator in place in a non-operating position;

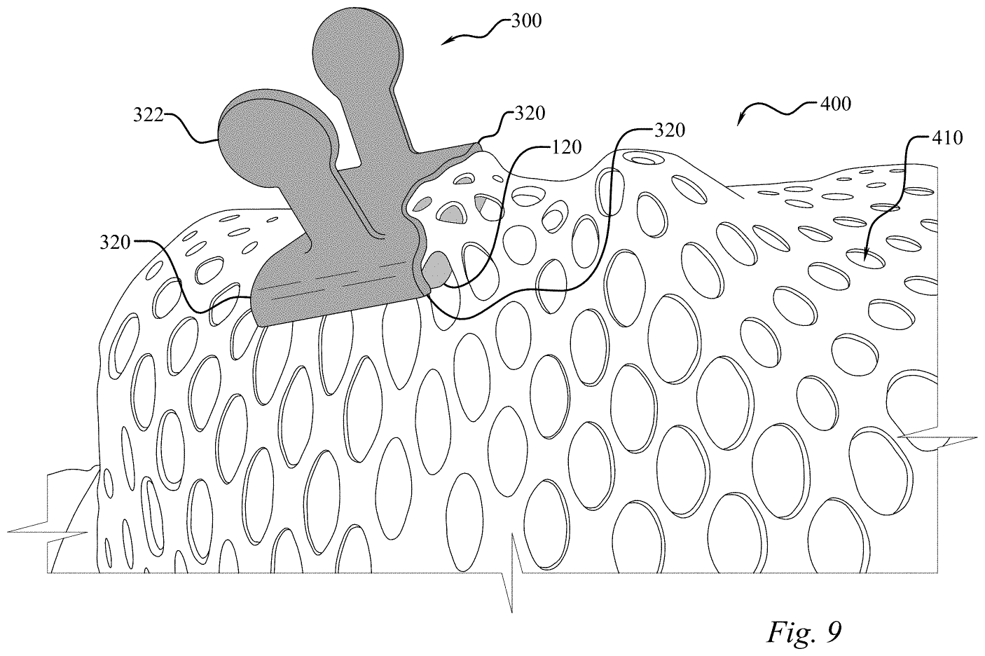

FIG. 9 is a further embodiment of the device of FIG. 1, further showing a digital manipulator in place in an operating position; and

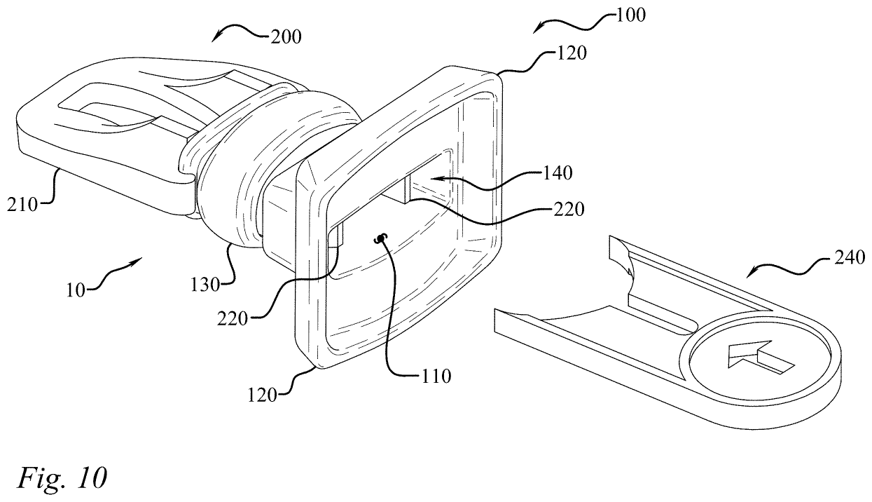

FIG. 10 is a further embodiment of the device of FIG. 1, showing a release tool in a disengaged state relative to a bite block and tongue diverter; and

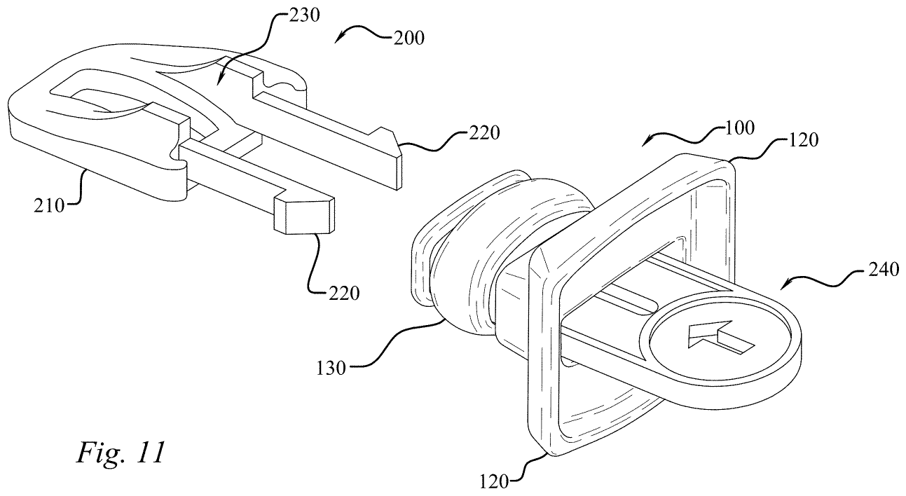

FIG. 11 is a further embodiment of the device of FIG. 1, showing a release tool in an engaged state relative to a bite block, having released a tongue diverter.

These illustrations are provided to assist in the understanding of the exemplary embodiments of the method of forming an immobilization system having a bite block stabilization feature and materials related thereto described in more detail below and should not be construed as unduly limiting the specification. In particular, the relative spacing, positioning, sizing and dimensions of the various elements illustrated in the drawings may not be drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

What is claimed then, as seen in FIGS. 1-10, is an immobilization system (10) for highly reproducible patient positioning. In one embodiment, seen well in FIGS. 1, 2, 7 and 9, the system (10) may include a bite block immobilization system (10) having a bite block (100) having a dental interface (130) releasably engageable with a plurality of teeth of a patient, and a bite block-retention mask interface (120) releasably engageable with a retention mask (400). The dental interface (130) may include a moldable dental impression putty in some embodiments, while in others, may include a moldable thermoplastic ring. The dental interface may be supplied with the system (10), or may be added at the time of use by a user. The dental interface (130) releasably engages the teeth of a patient and allows very highly accurate repositioning during multiple procedures.

The retention mask (400), seen well in FIG. 9, may be releasably and memorably moldable to the contours of a face of the patient. Such an embodiment, seen well in FIGS. 8 and 9, may also have a retainer means (300) cooperating with the bite block (100) and the retention mask (400) to releasably secure the bite block (100) and the retention mask (400). As would be known to one skilled in the art, the retention mask (400) may be secured to a backing board by any feasible releasable securing means for greater stability and reproducibility.

In some embodiments, seen well in FIGS. 7 and 9, the retention mask (400) may have a plurality of mask fenestrations (410). In other embodiments, seen well in FIGS. 1-3, the bite block (100) may have an integral bite block airway passage (110). This allows a patient to have unobstructed breathing both during molding of the retention mask (400) and during subsequent use of the system (10).

In yet another series of embodiments, the bite block (100), seen well in FIGS. 1-4 and 8, releasably cooperates at a tongue diverter receiver (140) with a tongue diverter (200), while in others, seen well in FIGS. 2-3, the tongue diverter receiver (140) may be releasably engageable with a tongue diverter retainer (220) to releasably engage the tongue diverter (200) and the bite block (100).

The tongue diverter (200) may further include as seen well in FIG. 4, a diverter airway (230) and may also include, a seen well in FIGS. 3-5, a lingual interface (210). The lingual interface (200), as would be known by one skilled in the art, may divert the tongue of a patient in a lateral, cephalic, or caudal direction, or in some combination of multiple directions.

In various embodiments, seen by way of example and not limitation only in FIGS. 8-9, the retainer means (300) may be releasably engageable with bite block (100) and the retention mask (400) by means of an interface retainer (320) releasable securing the bite block (100) to the retention mask (400). Such an interface retainer (320) may be a humanly releasable digital manipulator (322). In still other embodiments, seen well in FIGS. 10-11, a tongue diverter removal tool (240) may be releasably engageable with the tongue diverter receiver (140) to releasably disengage the tongue diverter (200) and the bite block (100).

In another series of embodiments, a bite block immobilization system (10) may include a bite block (100) having an integral bite block airway passage (110), and a dental interface (130) releasably engageable with a plurality of teeth of a patient. The bite block (100) may be releasably cooperating at a tongue diverter receiver (140) with a tongue diverter (200) having a lingual interface (210) and a diverter airway (230). Such a tongue diverter receiver (140) may be releasably engageable with a tongue diverter retainer (220) to releasably engage the tongue diverter (200) and the bite block (100). Such embodiments may also include a bite block-retention mask interface (120) releasably engageable with a retention mask (400), and such a retention mask (400) may be being releasably and memorably moldable to the contours of a face of the patient. Further, there may be a retainer means (300) cooperating with the bite block (100) and the retention mask (400) to releasably secure the bite block (100) and the retention mask (400).

In some embodiments, the tongue diverter (200) may further include a lingual interface (210) that may divert the tongue of a patient in a lateral, cephalic, or caudal direction, or allow a diversion in multiple axes of direction. In other embodiments, the retainer means (300) may be releasably engageable with a bite block (100) and the retention mask (400) by means of an interface retainer (320) releasable securing the bite block (100) to the retention mask (400), and the interface retainer (320) may be a humanly releasable digital manipulator (322). This obviates the need for a user to maintain pressure over the retention mask (400) while the retention mask (400) is converted from a more flexible, to a less flexible, state, most commonly by allowing the retention mask (400) to thermally cool.

One skilled in the art would recognize the most optimal methods of using the immobilization system. Steps for use might include; placing a bite block (100) between the maxilla and mandible of a human patient, and then placing a thermally moldable retention mask (400), in a moldable state, over at least a portion of the face of the human patient. Next, a user might secure the retention mask (400) to the bite-block (100) with a releasably engageable retainer means (300). Lastly, a user might observe or facilitate the conversion of the retention mask (400) to a fixed state, which could occur by any known method of converting the retention mask (400) from a flexible to a more rigid state, such as thermal hardening, and then, disengaging the releasably engageable retainer means (300).

Securing the releasable engageable retainer means (300) to the bite-block could be accomplished by the application of digital human pressure, and a subsequent step of may include the step of disengaging the releasable engageable retainer means (300) to the bite-block by the application of digital human pressure.

Numerous alterations, modifications, and variations of the preferred embodiments disclosed herein will be apparent to those skilled in the art and they are all anticipated and contemplated to be within the spirit and scope of the disclosed specification. For example, although specific embodiments have been described in detail, those with skill in the art will understand that the preceding embodiments and variations can be modified to incorporate various types of substitute and or additional or alternative materials, relative arrangement of elements, order of steps and additional steps, and dimensional configurations. Accordingly, even though only few variations of the method and products are described herein, it is to be understood that the practice of such additional modifications and variations and the equivalents thereof, are within the spirit and scope of the method and products as defined in the following claims. The corresponding structures, materials, acts, and equivalents of all means or step plus function elements in the claims below are intended to include any structure, material, or acts for performing the functions in combination with other claimed elements as specifically claimed.

I claim:

1. A bite block immobilization system (10) comprising:
a bite block (100) having a dental interface (130) releasably engageable with a plurality of teeth of a patient, and a bite block-retention mask interface (120) releasably engageable with a retention mask (400);
the retention mask (400) being releasably and memorably moldable to the contours of a face of the patient; and
a retainer means (300) cooperating with the bite block (100) and the retention mask (400) to releasably secure the bite block (100) and the retention mask (400).

2. The device according to claim 1, wherein the retention mask (400) has a plurality of mask fenestrations (410).

3. The device according to claim 1, wherein the bite block (100) has an integral bite block airway passage (110).

4. The device according to claim 1, wherein the bite block (100) releasably cooperates at a tongue diverter receiver (140) with a tongue diverter (200).

5. The device according to claim 1, wherein the dental interface (130) further comprises a dental impression putty.

6. The device according to claim 1, wherein the dental interface (130) further comprises a moldable thermoplastic ring.

7. The device according to claim 4, wherein the tongue diverter receiver (140) is releasably engageable with a tongue diverter retainer (220) to releasably engage the tongue diverter (200) and the bite block (100).

8. The device according to claim 4, wherein the tongue diverter (200) further comprises a diverter airway (230).

9. The device according to claim 4, wherein the tongue diverter (200) further comprises a lingual interface (210).

10. The device according to claim 9, wherein the lingual interface (200) adjustably diverts the tongue of a patient in a direction selected from a lateral, cephalic, caudal, and a combination of such directions, relative to the patient.

11. The device according to claim 1, wherein the retainer means (300) is releasably engageable with bite block (100) and the retention mask (400) by means of an interface retainer (320) releasable securing the bite block (100) to the retention mask (400).

12. The device according to claim 1, wherein the interface retainer (320) is a humanly releasable digital manipulator (322).

13. The device according to claim 4, wherein a tongue diverter removal tool (240) is releasably engageable with the tongue diverter receiver (140) to releasably disengage the tongue diverter (200) and the bite block (100).

14. A bite block immobilization system (10) comprising:
a bite block (100) having an integral bite block airway passage (110), a dental interface (130) releasably engageable with a plurality of teeth of a patient, wherein the bite block (100) releasably cooperates at a tongue diverter receiver (140) with a tongue diverter (200) having a lingual interface (210) and a diverter airway (230), and wherein the tongue diverter receiver (140) is releasably engageable with a tongue diverter retainer (220) to releasably engage the tongue diverter (200) and the bite block (100);
a bite block-retention mask interface (120) releasably engageable with a retention mask (400), releasably and memorably moldable to the contours of a face of the patient; and
a retainer means (300) cooperating with the bite block (100) and the retention mask (400) to releasably secure the bite block (100) and the retention mask (400).

15. The device according to claim 14, wherein the tongue diverter (200) further comprises a lingual interface (210) adjustably diverting the tongue of a patient in a direction selected from a lateral, cephalic, caudal, and a combination of such directions, relative to the patient.

16. The device according to claim 14, wherein the retainer means (300) is releasably engageable with the bite block (100) and the retention mask (400) by means of an interface retainer (320) releasable securing the bite block (100) to the retention mask (400).

17. The device according to claim 16, wherein the interface retainer (320) is a humanly releasable digital manipulator (322).

18. A method for forming a bite block immobilization system (10) comprising the steps of:
1. placing a bite block (100) between the maxilla and mandible of a human patient;
2. placing a thermally moldable retention mask (400), in a moldable state, over at least a portion of a face of the human patient;
3. securing the retention mask (400) to the bite-block (100) with a releasably engageable retainer means (300);
4. facilitating the conversion of the retention mask (400) to a fixed state; and
5. disengaging the releasably engageable retainer means (300).

19. The method of claim 18, further comprising the step of securing the releasable engageable retainer means (300) to the bite-block by the application of digital human pressure.

20. The method of claim 18, further comprising the step of disengaging the releasable engageable retainer means (300) to the bite-block by the application of digital human pressure.
